(19) United States
(12) Patent Application Publication
(10) Pub. No.: US 2011/0129506 A1
(43) Pub. Date: Jun. 2, 2011

(54) Colouring using pearlescent pigments in the food and pharmaceutical sectors

(76) Inventors: Ralf SCHWEINFURTH, Wiesloch (DE); Uta Hillgärtner, Darmstadt (DE)

(21) Appl. No.: 12/951,498

(22) Filed: Nov. 22, 2010

Related U.S. Application Data
(63) Continuation of application No. 11/775,579, filed on Jul. 10, 2007, now abandoned.
Application No. 11/775,579 is a continuation of application No. 09/743,758, filed on Jan. 16, 2001, now abandoned.

(30) Foreign Application Priority Data
Jul. 16, 1998 (DE) 198 31 869.3

(51) Int. Cl. A61K 47/02; A23L 1/275; A61K 47/04; A61K 9/00

(52) U.S. Cl. 424/400; 426/540; 426/250; 514/769; 514/770

(57) ABSTRACT

The present invention relates to the use of titanium dioxide pigments and/or iron oxide pigments based on platy substrates for colouring food products and pharmaceutical products.

The present invention relates to the use of titanium dioxide pigments and/or iron oxide pigments based on platy substrates for colouring food products and pharmaceutical products.

In addition to their functional applications, pearlescent pigments and interference pigments are also increasingly being used for the visual enhancement of products, for example in cosmetics, since fine colours and effects give rise to pleasing subjective impressions on the part of the viewer and consumer. Since, in the production of pearlescent pigments, for example for decorative cosmetics, the strictest requirements are made as to the purity and quality of the pigments, pearlescent pigments should also be usable in the food sector for improving the colouring effect or for colouring.

The object of the present invention is therefore to expand the palette of the previously known colours in the colouring of food products and pharmaceutical products using pearlescent pigments or interference pigments, as the result of which the products experience an additional sensorially perceptible enhancement. The visual enhancement is valuable in particular for pharmaceutical products, since a clearer differentiation is made possible between differently coloured tablets, dragees, etc.

Surprisingly, it has now been found that for the visual enhancement of food products and pharmaceutical products, platy substrates coated with titanium dioxide and/or iron oxide are outstandingly suitable. Iron oxide pigment is taken to mean platy substrates coated not only with Fe₂O₃ but also with Fe₃O₄. In particular, the combination of TiO₂ pigments and/or Fe₃O₄ pigments with natural or nature-identical dyes, colour pigments or colorant fruit extracts and plant extracts give the food product an interesting new colour. By means of the colouring of food products, simultaneously desires for novel variants and nuances in colours as the result of novel trends in fashion can be taken into account.

The invention thus relates to the use of titanium dioxide pigments and/or iron oxide pigments based on platy substrates for colouring food products and pharmaceutical products.

The coloured foods and pharmaceutical products are distinguished by a novel colouring effect which is based on light refraction by the pearlescent pigments and causes pleasant subjective impressions in the viewer and consumer. This optical effect is not possible using the colorants which are currently permitted in the food sector. In contrast to colour pigments which are permitted in the food sector, for example vegetable carbon E153, the titanium dioxide pigments and iron oxide pigments based on platy substrates may be dispersed very readily into the medium to be pigmented. Furthermore, the products thus coloured are distinguished by an increased protection from light and moisture. Vitamin preparations in particular have a longer shelf life. In the case of the colouring of tablets, in many cases a delayed release of active compounds has been observed.

It has been found that even at very low amounts of titanium oxide pigments and/or iron oxide pigments novel interesting colours and simultaneously novel properties can be imparted to the foods or pharmaceutical products. Outstanding results are achieved even when the product is coloured with 0.005 to 15.0% by weight, preferably 0.01 to 6.0% by weight, in particular 0.1 to 2.0% by weight of pearlescent pigment, based on the product.

In the event that the pigment is added directly to the food product or pharmaceutical product during manufacture, the amount of pigment used is preferably 0.005 to 4% by weight. In the case of the surface treatment of foods or tablets, the application range is 0.02 to 15.0% by weight, preferably 0.5 to 6.0% by weight, based on the colorant solution or coating solution.

The black iron oxide pigments used are magnetite-coated natural or synthetic mica platelets, talc, kaolin, SiO₂ platelets or TiO₂ platelets. Particularly preferably, finely divided iron oxide pigments are used to colour the products, preferably having a particle size in the range from 0.01 to 200 μm, in particular from 0.1 to 100 μm. Black pearlescent pigments of this type are disclosed, for example, by the patents and patent applications P 23 13 331, P 36 17 430 and JP 90-246314. These pigments are commercially available, for example, under the trademark Candurin® Black Fine from Merck KGaA, Darmstadt.

TiO₂ pigments and Fe₂O₃ pigments based on platy substrates, for example natural and synthetic mica, glass platelets, TiO₂ platelets, SiO₂ platelets and Al₂O₃ platelets are disclosed, for example, by German patents and patent applications DE 14 67 468, DE 19 59 998, DE 20 09 566, DE 22 14 545, DE 22 15 191, DE 22 44 298, DE 23 13 331, DE 25 22 572, DE 31 37 808, DE 31 37 809, DE 31 51 343, DE 31 51 354, DE 31 51 355, DE 32 11 602, DE 32 53 017, WO 93/08237, DE 196 18 564 and EP 0 763 573. The substrates in these patents are coated with one, two, three, four, five or more metal oxide layers. In particular, preference is given to pigments which comprise one or two layers of TiO₂ and/or Fe₂O₃. The particle sizes of the pigments are preferably ≦200 μm, in particular ≦100 μm.

By adding pearlescent pigments and interference pigments which are permitted for the food sector, and/or natural/nature-identical dyes, colour pigments or colorant fruit extracts and plant extracts, the colouring effect of the titanium dioxide pigments and/or iron oxide pigments can be intensified in the product and simultaneously novel colouring effects can be achieved.

In particular, the colouring of food products with a pigment mixture consisting of Fe₃O₄ pigments with pearlescent pigments and/or interference pigments based on mica platelets, Al₂O₃ platelets, SiO₂ platelets or TiO₂ platelets which are coated with TiO₂ and/or Fe₂O₃, imparts interesting colouring effects to the products. Very particular preference is given to pigment mixtures comprising an Fe₃O₄ pigment based on mica.

Combining Fe₃O₄ pigments with other pearlescent pigments such as gold pigments, silver pigments or interference pigments intensifies the respective colouring effect of the pigments. This synergy extends considerably the colouring possibilities of the product to be pigmented, without other natural or nature-identical dyes having to be additionally used.

The pearlescent pigments and interference pigments permitted for the food sector are commercially available, for example, under the trademark Candurin® from Merck KGaA.

The total concentration of all pigments in the product to be pigmented should also not exceed 12% by weight, based on the product. The concentration is generally dependent on the specific application. The mixing ratio of the TiO₂ pigments or Fe₃O₄ pigments with a further pigment component depends on the desired effect and is generally 20:1 to 1:20, preferably 5:1, in particular 1:1. The pigment component can be one or more pearlescent pigments or interference pigments. In particular preference is given to TiO₂-mica pigments, Fe₂O₃-mica pigments and TiO₂- and Fe₂O₃-coated mica pigments. In addition, preference is given to TiO₂- and/or Fe₂O₃-coated or uncoated SiO₂ platelets or TiO₂ platelets.

The colouring effect in food products and pharmaceutical products is improved in particular when black iron oxide pigments are combined with gold pigments, silver pigments and interference pigments based on TiO₂- or TiO₂/Fe₂O₃-coated mica pigments. The content of Fe₃O₄ pigments in the product should, in this preferred embodiment, preferably be 0.005-2% by weight.

In addition to the pearlescent pigments and/or interference pigments, all natural or nature-identical dyes known to those skilled in the art can be added as further colouring component to the titanium dioxide pigments and/or iron oxide pigments. In particular those which may be mentioned here are: E 101, E 104, E 110, E 124, E 131, E 132, E 140, E 141, E 151, E 160a.

In addition, other colouring pigments can be added to the platy pearlescent pigments, for example E 171, E 172, E 153.

The content of dyes based on the product is in the range from 0.5 to 25% by weight. Also, fruit extracts and plant extracts can be used as dye, for example carrot juice, beetroot juice, elderberry juice, hibiscus juice, paprika extract, aronia extract.

The pharmaceutical products and food products are coloured by adding the titanium dioxide pigment and/or iron oxide pigment to the product to be coloured alone or in combination with other pigments or colorants in the desired quantitative ratios, simultaneously or successively, during or after their production. Laborious grinding and dispersion of the pigments is not necessary.

Products suitable for colouring which may be mentioned are in particular coatings on all types of foods, in particular pigmented sugar coatings and shellac coatings (alcoholic and aqueous), coatings containing oils and waxes, containing gum arabic and cellulose types (e.g. HPMC=hydroxypropyl methyl cellulose), incorporation into or application onto confectionery, cake decorations, compressed tablets, pan-coated products, chewing gums, gums, fondant products, marzipan products, fillings, cocoa icings and fat icings, chocolate and chocolate-containing products, ice cream, cereals, snack products, coating compositions, gateaux presentation plates, hundreds and thousands, sugar crystals, jelly and gelatin products, sweets, liquorice, icing, candyfloss, fat, sugar and baker's cream compositions, puddings, desserts, flan glazing, cold sweet soups, beverages containing stabilizing additives such as carboxy methyl cellulose, acidified and non-acidified milk products such as quark, yoghurt, cheese, cheese rinds, sausage casings, etc.

A further large field of use is the pharmaceutical and OTC sector for colouring tablets, gelatin capsules, sugar-coated tablets, ointments, cough syrup, etc. In combination with customary coatings such as polymethacrylates and celluloses, for example HPMC, the pigments can be used in many ways for colouring.

In the case of pan-coated or otherwise coated food products and pharmaceutical products, it is possible to combine the titanium dioxide pigments and iron oxide pigments with flavourings (powdered flavourings or liquid flavourings) and/or with sweeteners, for example aspartame, in order to accentuate the visual effect also in terms of flavour.

The invention thus relates to all formulations from the food sector and pharmaceutical sector comprising the titanium dioxide pigment and/or iron oxide pigment alone or in combination with other pigments/pigment mixtures or dyes (natural or nature-identical) as colorants.

The examples below are intended to describe the invention without restricting it, however.

### EXAMPLES

### Example 1

### Hard Caramel Manufacturing Details

### 1. Example Formula Containing Isomalt

| | | Obtainable from: |
|---|---|---|
| Isomalt Merck | 75% | Palatinit GmbH, |
| Patent GmbH | | Mannheim |
| Water | 24.005% | |
| Aspartame | 0.0075% | Worlée, Hamburg |
| Acesulfame-K | 0.0075% | Nutrinova, |
| | | Frankfurt a.M. |
| Candurin ® Silver | 0.144% (0.2% based | Merck KGaA, |
| Sheen*¹ | on the pouring mass) | Darmstadt |
| Candurin ® Black | 0.036% (0.05% based | Merck KGaA, |
| Fine*² | on the pouring mass) | Darmstadt |
| Citric acid | 0.5% | Merck KGaA, |
| | | Darmstadt |
| Flavouring | 0.3% (peach 9/030307) | Dragoco, Holzminden |

*¹(TiO₂-mica pigment of particle size 5-25 μm)
*²(Fe₃O₄mica pigment of particle size <15 μm)

Water is heated with Isomalt M to 165° C. and the solution is then allowed to cool to 145° C. After addition of citric acid, aspartame, Acesulfame-K, the pigments Candurin® Black Fine and Candurin® Silver Sheen and the flavouring are stirred in. Finally the hot mixture is poured into greased moulds using a pouring funnel. The hard caramels are allowed to cool for 1 hour.

Further embodiments:
It is frequently advisable to dissolve the ingredients in advance using some of the water in order to avoid lump formation.
The pigments Candurin® Silver Sheen and Candurin® Black Fine can also be heated directly with the water and Isomalt. No loss of colour occurs.

### 2. Example Formula Containing Isomalt

| | | Obtainable from: |
|---|---|---|
| Isomalt Merck | 75% | Palatinit GmbH, |
| Patent GmbH | | Mannheim |
| Water | 24.041% | |
| Aspartame | 0.0075% | Worlée, Hamburg |
| Acesulfame-K | 0.0075% | Nutrinova, |
| | | Frankfurt a.M. |
| Candurin ® | 0.144% (0.2% based | Merck KGaA, |
| Honeygold*³ | on the pouring mass) | Darmstadt |
| Citric acid | 0.5% | Merck KGaA, |
| | | Darmstadt |
| Flavouring | 0.3% (peach 9/030307) | Dragoco, Holzminden |

*³(TiO₂/Fe₂O₃-mica pigment of particle size 5-25 μm)

Water is heated with Isomalt M to 165° C. and the solution is then allowed to cool to 145° C. After addition of citric acid, aspartame, Acesulfame-K, Candurin® Honeygold and the flavouring are stirred in. Finally, the hot mass is poured into greased moulds using a pouring funnel. The hard caramels are allowed to cool for 1 hour.

Further embodiments:
Frequently it is advisable to dissolve the ingredients in advance using some of the water in order to prevent lump formation.
The pigment Candurin® Honeygold can also be heated directly with the water and Isomalt.

### 3. Example Formula Containing Sugar

| | | Obtainable from: |
|---|---|---|
| Sugar | 41% | Südzucker |
| Water | 17.077% | |
| Glucose syrup | 41% C* Sweet | Cerestar, Krefeld |
| Candurin ® Silver | 0.082% (0.1% based | Merck KGaA, |
| Sheen*¹ | on the pouring mass) | Darmstadt |
| Candurin ® Black | 0.041% (0.05% based | Merck KGaA, |
| Fine*² | on the pouring mass) | Darmstadt |
| E 104 1:100 dilution | 0.4% Sikovit | BASF, Ludwigshafen |
| Flavouring | 0.4% (banana 9/030388) | Dragoco, Holzminden |

*¹(TiO₂-mica pigment of particle size 5-25 μm)
*²(Fe₃O₄-mica pigment of particle size <15 μm)

The sugar is heated with the water to 100° C. and then glucose syrup is added. The solution is then heated to 145° C. After addition of Candurin® Silver Sheen, Candurin® Black Fine, dye solution and the flavouring, the caramel solution is poured into greased moulds using a pouring funnel. Finally, the mixture is allowed to cool for two hours. The Candurin® pigments can be either mixed with the sugar or added in a mixture with the glucose syrup. This variant contains no acid, since this would make the caramelization too intense.

### 4. Example Formula Containing Sugar

| | | Obtainable from: |
|---|---|---|
| Sugar | 41% | Südzucker |
| Water | 17.118% | |
| Glucose syrup | 41% C* Sweet | Cerestar, Krefeld |
| Candurin ® Wine | 0.082% (0.1% based | Merck KGaA, |
| Red*⁴ | on the pouring mass) | Darmstadt |
| E 104 1:100 dilution | 0.4% Sikovit | BASF, Ludwigshafen |
| Flavouring | 0.4% (banana 9/030388) | Dragoco, Holzminden |

*⁴(Fe₂O₃-mica pigment of particle size 10-60 μm)

The sugar is heated with the water to 100° C. and then glucose syrup is added. The solution is then heated to 145° C. After addition of Candurin® Wine Red, dye solution and the flavouring, the caramel solution is poured into greased moulds using a pouring funnel. Finally, the mixture is allowed to cool for two hours. The Candurin® pigment can be either mixed with the sugar or added as a mixture with the glucose syrup. This variant contains no acid, since this would make the caramelization too intense.

### Example 2

### Manufacture of Gelatin Articles

### 1. Example Formula

| | | Obtainable from: |
|---|---|---|
| Water | 10.48% | |
| Sugar | 31.45% | Südzucker |
| Glucose syrup | 31.45% C* Sweet | Cerestar, Krefeld |
| Candurin ® Wine | 0.38% (0.4% based | Merck KGaA, |
| Red | on the pouring mass) | Darmstadt |
| Citric acid 1:1 | 2.51% | Merck KGaA, |
| dilution | | Darmstadt |
| Gelatin | 7.86% 260 Bloom | DGF, Eberbach |
| Water | 15.748% | |
| Flavouring | 0.122% (blackcurrant | Dragoco, |
| | 9/695750) | Holzminden |

The gelatin is first softened with twice the amount of water at 60° C. Sugar and water are heated to 100° C., then the glucose syrup is added. The mixture is heated further to 120° C. and is then allowed to cool to approximately 85° C. The Candurin® pigment, the citric acid, the flavouring and the gelatin solution are stirred in, and the deaerated gelatin mixture is charged into greased moulds using the pouring funnel. The product is allowed to cool for approximately 16 hours.

Further embodiments:
The Candurin® pigment can here again be already mixed directly with the sugar or introduced together with the glucose syrup.
Instead of pouring into moulds, the traditional method using negative moulds in moulding powder can be used in this case to produce gelatin articles.

### 2. Example Formula

| | | Obtainable from: |
|---|---|---|
| Water | 10.508% | |
| Sugar | 31.45% | Südzucker |
| Glucose syrup | 31.45% C* Sweet | Cerestar, Krefeld |
| Candurin ® Blueberry | 0.38% (0.4% based | Merck KGaA, |
| Sugar*⁵ | on the pouring mass) | Darmstadt |
| E 153 (vegetable | 0.038% (0.04% based | Dr. Marcus |
| carbon/Carbon | on the pouring mass) | |
| medicinales) | | |
| Citric acid 1:1 | 2.51% | Merck KGaA, |
| dilution | | Darmstadt |
| Gelatin | 7.86% 260 Bloom | DGF, Eberbach |
| Water | 15.682% | |
| Flavouring | 0.122% (blackcurrant | Dragoco, |
| | 9/695750) | Holzminden |

*⁵(TiO₂-mica pigment of particle size 10-60 μm)

The gelatin is first softened with the same amount of water at 60° C. Sugar and water are heated to 100° C., then the glucose syrup is added. The mixture is heated further to 120° C. and is then allowed to cool to approximately 85° C. The Candurin® pigment, the citric acid, the flavouring and the gelatin solution are stirred in, and the deaerated gelatin mixture is charged into greased moulds using the pouring funnel. The product is allowed to cool for approximately 16 hours.

Further embodiments:
The Candurin® pigment can here again be already mixed directly with the sugar or introduced together with the glucose syrup.
Instead of pouring into moulds, the traditional method using negative moulds in moulding powder can be used in this case to produce gelatin articles.

### Example 3

### Dragees

a) Cores to be Coloured: White Dragee Cores (Liquorice Rods with a Hard Sugar Coating)

Solution for coating the dragees:

| | | Obtainable from: |
|---|---|---|
| alcoholic shellac solution | 95.62% | Wolff & Olsen |
| Candurin ® Silver Lustre*¹ | 4.00% | Merck KGaA, Darmstadt |
| Candurin ® Black Fine*² | 0.38% | Merck KGaA, Darmstadt |

*¹(TiO₂-mica pigment of particle size 10-60 μm)
*²(Fe₃O₄-mica pigment of particle size <15 μm)

The Candurin® pigments are evenly distributed in the shellac and sprayed onto the dragees which are slowly rotating in the dragee pan. The dragees are continuously dried using cold air. The spraying is continued until the desired colour coverage is achieved. Finally, the cores are taken out of the pan and dried on racks for approximately 12 hours.

### b) Cores to be Coloured: Black Dragee Cores (Chewing Gum Balls) Solution for Coating the Dragees:

| | | Obtainable from: |
|---|---|---|
| alcoholic shellac solution | 95% | Kaul GmbH |
| Candurin ® Caramel* | 5% | Merck KGaA, Darmstadt |

*(Fe₂O₃-mica pigments of particle size 10-60 μm)

The Candurin® pigment is evenly distributed in the shellac and sprayed onto the dragees which are slowly rotating in the dragee pan. Spraying is continued until the desired colour coverage is achieved. Drying is performed continuously using cold air in order to prevent the cores from sticking together. Finally, the cores are taken out of the pan and dried on racks for approximately 12 hours.

c)—Coating Liquorice Nibs with a White Hard Sugar Shell

Cores to be coloured: liquorice nibs having a white hard sugar shell

Solution for coating the dragees:

| | | Obtainable from: |
|---|---|---|
| alcoholic shellac solution | 95.75% | Capol 425 Kaul GmbH |
| Candurin ® Buttergold*¹ | 4% | Merck KGaA, Darmstadt |
| Candurin ® Black Fine | 0.25% | Merck KGaA, Darmstadt |

*¹(TiO₂/Fe₂O₃-mica pigment of particle size 10-60 μm)

The cores are coated in a similar manner to Example 3 a).

d)—Pan-Coating of Viennese Nuts (White, Hard Sugar Shell with Hazelnut Core) Solution for Coating the Dragees:

| | | Obtainable from: |
|---|---|---|
| alcoholic shellac solution | 96.0% | Wolff & Olsen |
| Candurin ® Silver Lustre* | 4% | Merck KGaA, Darmstadt |

*(TiO₂-mica pigment of particle size 10-60 μm)

The Candurin® pigment is distributed evenly in the shellac solution. It is applied in a similar manner to Example 3 a).

e)—Starting Material: White Chocolate Drops

An aqueous Candurin®/HPMC solution is used for the spray application.
The Candurin® pigments are stirred into water. Then, if appropriate, additional dyes, flavourings or sweeteners are then added. Finally, the film-forming agent (HPMC) is added to the suspension. As a result of the increasing viscosity, the stirrer speed must also be correspondingly increased. After approximately 40-60 minutes, the HPMC is completely dissolved and the solution can then be sprayed onto the dragees.
The spray application can be performed in the pan-coating drum or in customary coating systems, with care being taken to ensure an appropriately matched continuous drying air stream (temperatures/volumes).
As soon as the desired colouring effect is achieved, the dragees are discharged. They are then dried on racks.

### Spray Solution:

| | | |
|---|---|---|
| Sepifilm Lp10 | 6.0% | Seppic |
| Candurin ® paprika* | 5.0% | Merck KGaA, Darmstadt |
| Flavouring | 0.5% | Dragoco, Holzminden |
| | (vanilla 9/024233) | |
| Water | 88.5% | |

*(Fe₂O₃-mica pigment of particle size 10-60 μm)

### f) Starting Material: White Sugar-Pan-Coated Almonds

The spray solution is prepared and applied as in e)

### Spray Solution:

| | | |
|---|---|---|
| Sepifilm Lp10 | 5.0% | Seppic |
| Candurin ® Silver Lustre* | 4.0% | Merck KGaA, Darmstadt |
| Sepisperse M5062 | 1.0% | Seppic |
| Water | 90.0% | |

*(TiO₂-mica pigment of particle size 10-60 μm)

### g) Starting Material: White Sugar-Pan-Coated Almonds

The spray solution is prepared and applied as in e)

### Spray Solution:

| | | |
|---|---|---|
| Sepifilm Lp10 | 6.0% | Seppic |
| Candurin ® Silver Lustre* | 4.0% | Merck KGaA, Darmstadt |
| E153 | 0.2% | Dr. Marcus |
| Water | 89.8% | |

*(TiO₂-mica pigment of particle size 10-60 μm)

### h) Starting Material: Red Sugar-Pan-Coated Almonds

An aqueous Candurin®/gum arabic solution is used for the spray application.

The Candurin® pigments are stirred into the gum arabic solution and are then sprayed onto the dragees rotating in the pan-coating drum. Drying should be performed continuously here using cold air. When the desired colouring effect is achieved, the spraying operation is terminated. The coloured dragees can then further be coated with a shellac film to avoid sticking together.

### Spray Solution:

| | | |
|---|---|---|
| Gum arabic solution | 82.0% | Kaul |
| Candurin ® Wine Red* | 8.0% | Merck KGaA, Darmstadt |
| Water | 10.0% | |

*(Fe₂O₃-mica pigment of particle size 10-60 μm)

### Example 4

### Jelly Dessert

| | | Obtainable from: |
|---|---|---|
| Calcium lactate | 0.05% | Merck KGaA |
| Genugel LC4N | 0.6% | Hercules |
| Tetrasodium phosphate | 0.15% | Merck KGaA |
| Citric acid crystalline | 0.38% | Merck KGaA |
| gellan | 0.06% | Kelco |
| Tripotassium phosphate | 0.05% | Merck KGaA |
| Sucrose | 15% | Südzucker |
| Candurin ® Silver Sparkle*¹ | 0.025% | Merck KGaA |
| Flavouring and colour optional | | |
| Water | 83.685% | |

*¹(TiO₂-mica pigment of particle size 20-150 μm)

All components are heated to 95° C. and kept at this temperature for 3-5 minutes with stifling. They are then allowed to cool with gentle stirring to 40-45° C. The jelly dessert is then poured into moulds and if necessary it is cooled.

### Pharmaceutical Applications:

### Example 5

### a) Initial Weight 1 kg of White Tablets d=8 mm, G=200 mg

### Solution for Film Coating:

| | | |
|---|---|---|
| 6% | Sepifilm Lp10 | Seppic |
| | (Mixture of hydroxypropyl methyl | |
| | cellulose, stearic acid and | |
| | microcrystalline cellulose) | |
| 5% | Candurin ® Caramel* | Merck KGaA, Darmstadt |
| 89% | Water | |

*(Fe₂O₃-mica pigment of particle size 10-60 μm)

Total amount applied: 200 g

This corresponds to 1.2 mg of polymer/cm² of tablet surface area

### b) Initial Weight 1 kg of White Tablets d=8 mm, G=200 mg

### Solution for Film Coating (100 g):

| | | |
|---|---|---|
| 6% | Sepifilm Lp10 | Seppic |
| 4% | Candurin ® Silver Lustre* | Merck KGaA |
| 1% | Sepisperse M5062 | Seppic |
| | (Dye paste: TiO₂, natural or nature-identical dyes, | |
| | stabilizer) | |
| 89% | Water | |

*(TiO₂-mica pigment of particle size 10-60 μm)

Total amount applied: 200 g

This corresponds to 1.2 mg of polymer/cm² of tablet surface area

### Production of the Film-Coating Solution:

The Candurin® pigments are stirred into water. If appropriate, additional dyes are then added. Finally, the film-forming agent (HPMC) is dispersed in the suspension. The increasing viscosity necessitates the stirrer viscosity also being correspondingly increased. After approximately 40-60 minutes, the HPMC is completely dissolved and the solution can then be sprayed onto the tablets.
The spray application is made using a customary standard coating process.

### Sugar Confectionery

### Example 6

### a) Liquorice Products

Raw material: extruded liquorice products

The oiled liquorice products are sprayed with a Candurin®/shellac solution in a pan-coating drum. At the same time they are dried using cold air. As soon as the desired colouring effect is achieved, application is halted and the coloured liquorice products are discharged from the pan.

### Spray Solution:

| | | |
|---|---|---|
| Alcoholic shellac solution | 97% | Kaul |
| Candurin ® Kiwi Sugar* | 3% | Merck KGaA, Darmstadt |

*(TiO₂-mica pigment of particle size 10-60 μm)

### b) Menthol Pastilles

Raw material: menthol pastilles

A Candurin®/shellac solution (aqueous) is sprayed onto the rotating pastilles in the pan-coating drum. Drying is performed continuously with warm air here. As soon as the desired colouring effect is achieved, application is terminated and the coloured pastilles are discharged from the pan.

### Spray Solution:

| | | |
|---|---|---|
| Alcoholic shellac solution | 96% | Warner Jenkinson |
| Candurin ® Silver Lustre* | 4% | Merck KGaA, Darmstadt |

*(TiO₂-mica pigment of particle size 10-60 μm)

### c) Marzipan Roses (Cake Decoration)

Raw material: red marzipan roses

The cake decorations are sprayed with a Candurin®/shellac solution until the desired colour application is achieved. Subsequent drying with cold air is possible.

### Spray Solution:

| | | |
|---|---|---|
| Alcoholic shellac solution | 97% | Wolff & Olsen |
| Candurin ® Blueberry Sugar* | 3% | Merck KGaA, Darmstadt |

*(TiO₂-mica pigment of particle size 10-60 μm)

### d) Sherbet Sweets

Raw material: sherbet sweets, white

The sherbet sweets are sprayed with a Candurin®/shellac solution until the desired colour application is achieved. Subsequent drying with cold air is possible.

### Spray Solution:

| | | |
|---|---|---|
| Alcoholic shellac solution | 94% | Kaul |
| Candurin ® Silver Sparkle* | 6% | Merck KGaA, Darmstadt |

*(TiO₂-mica pigment of particle size 20-150 μm)

1. Use of titanium dioxide pigments and/or iron oxide pigments based on platy substrates for colouring food products or pharmaceutical products.

2. Use of titanium dioxide pigments and/or iron oxide pigments according to claim 1, characterized in that the platy substrate is a mica platelet, Al₂O₃ platelet, TiO₂ platelet or SiO₂ platelet.

3. Use of titanium dioxide pigments and/or iron oxide pigments according to claim 1 characterized in that the iron oxide pigment is a platy substrate coated with Fe₂O₃ or Fe₃O₄.

4. Use of titanium dioxide pigments and/or iron oxide pigments according to claim 1, characterized in that the titanium dioxide pigment content and/or iron oxide pigment content in the food product or pharmaceutical product is 0.005 to 15% by weight.

5. Use of titanium dioxide pigments and/or iron oxide pigments according to claim 1, characterized in that the titanium dioxide pigment and the iron oxide pigment can be mixed in a ratio of 1:20 to 20:1.

6. Use of titanium dioxide pigments and/or iron oxide pigments according to claim 1, characterized in that titanium dioxide pigments and/or iron oxide pigments combined with one or more pearlescent pigments, coated or uncoated TiO₂ platelets, SiO₂ platelets, natural or nature-identical colorants, colour pigments or natural plant or fruit extracts are used.

7. Use of titanium dioxide pigments and/or iron oxide pigments according to claim 6, characterized in that the titanium dioxide pigments and/or iron oxide pigments are used in combination with one or more pearlescent pigments based on mica coated with TiO₂, Fe₂O₃ or a TiO₂/Fe₂O₃ mixture.

8. Use of titanium dioxide pigments and/or iron oxide pigments according to claim 6, characterized in that the iron oxide pigment is a mica platelet, SiO₂ platelet or TiO₂ platelet coated with Fe₃O₄.

9. Use of titanium dioxide pigments and/or iron oxide pigments according to claim 1, characterized in that the food product or pharmaceutical product is furnished with a coating of shellac, oils, waxes, gum arabic, celluloses, polymethacrylates or icing comprising titanium dioxide pigments and/or iron oxide pigments and if appropriate other pigments and/or colorants.

10. Process for the production of food products and pharmaceutical products coloured by titanium dioxide pigments and/or iron oxide pigments, characterized in that the titanium dioxide pigment and/or iron oxide pigment is added to the product to be coloured alone or in combination with other pigments or colorants in the desired quantitative ratios, simultaneously or successively, during or after their production.

11. Food products and pharmaceutical products comprising titanium dioxide pigments and/or iron oxide pigments based on platy substrates as colorant.
